(19) United States
(12) Patent Application Publication
(10) Pub. No.: US 2021/0390699 A1
(43) Pub. Date: Dec. 16, 2021

(54) IMAGE DISPLAY APPARATUS AND STORAGE MEDIUM

(71) Applicant: Konica Minolta, Inc., Tokyo (JP)

(72) Inventor: Kenji NAGAO, Tokorozawa-shi (JP)

(21) Appl. No.: 17/345,324

(22) Filed: Jun. 11, 2021

(30) Foreign Application Priority Data
Jun. 11, 2020 (JP) 2020-101302

(51) Int. Cl. G06T 7/00; G06T 3/40

(52) U.S. Cl. CPC G06T 7/0016; G06T 2207/30061; G06T 3/40

(57) ABSTRACT

An image display apparatus includes a display that displays multiple dynamic images and a hardware processor. The multiple dynamic images include at least two dynamic images and show a target region. The hardware processor enlarges/reduces at least one of the at least two dynamic images at an enlargement/reduction ratio that equalizes a size of the target region in a specific state among the at least two dynamic images, and causes the display to display the at least two dynamic images.

### CROSS-REFERENCE TO RELATED APPLICATIONS

The present invention claims priority under 35 U.S.C. § 119 to Japanese Patent Application No. 2020-101302 filed on Jun. 11, 2020 the entire content of which is incorporated herein by reference.

### BACKGROUND

### Technological Field

The present invention relates to an image display apparatus and a storage medium.

### Description of Related Art

In interpreting medical images, there is known a method of comparing a past image and a present image of a patient to make morphological diagnosis. These past and present images are enlarged at the same enlargement ratio and displayed for morphological diagnosis.

Recently, dynamic images have been used to make a diagnosis of the function of a target region of a subject (diagnosis based on the movement of the target region). A dynamic image consists of multiple frame images obtained by continuously capturing the dynamic state of the target region. For example, a diagnosis of the function of the lungs can be made on the basis of the change rate of the lungfield area in the dynamic image (change rate of the lung size from the maximal inspiratory level to the maximal expiratory level). However, general practitioners (doctors other than specialist in specific fields) or doctors who have performed only the morphological diagnosis by comparing two static images may not be familiar with using dynamic images and may find it difficult to make a diagnosis of the function with the dynamic images.

JP2019-103586A discloses a technique of adjusting cycles and/or phases of two or more dynamic images and displaying the adjusted dynamic images side by side for comparison.

### SUMMARY

In comparing a to-be-interpreted dynamic image of a patient with a dynamic image of another patient in a similar case, however, the technique in JP2019-103586A may not contribute to a pertinent function diagnosis because the size of the target region varies depending on the patient. For example, assume that two dynamic images of different patients are enlarged and displayed at the same enlargement ratio in the same way as two static images are compared, in order to visually compare absolute change amounts of the size of the expanding/contracting lungs between the two dynamic images. In the case, even if the change rate of the lungfield area is the same between the two dynamic images, the smaller lungs have a smaller absolute change amount in expansion/contraction than the larger lungs. As a result, an image interpreter may wrongly diagnose that the function of the smaller lungs is inferior to that of the larger lungs.

Objects of the present invention include allowing doctors to appropriately and smoothly make a diagnosis on the function of the target on the basis of multiple dynamic images displayed side by side for comparison.

To achieve at least one of the abovementioned objects, according to an aspect of the present invention, there is provided an image display apparatus including: a display that displays multiple dynamic images; and a hardware processor, wherein the multiple dynamic images include at least two dynamic images and show a target region, and the hardware processor enlarges/reduces at least one of the at least two dynamic images at an enlargement/reduction ratio that equalizes a size of the target region in a specific state among the at least two dynamic images, and causes the display to display the at least two dynamic images.

To achieve at least one of the abovementioned objects, according to another aspect of the present invention, there is provided a non-transitory computer-readable storage medium storing a program that causes a computer to function as a hardware processor that: among multiple dynamic images that are to be displayed on a display and that include at least two dynamic images and that show a target region, enlarges/reduces at least one of the at least two dynamic images at an enlargement/reduction ratio that equalizes a size of the target region in a specific state among the at least two dynamic images, and causes the display to display the at least two dynamic images.

### BRIEF DESCRIPTION OF THE DRAWINGS

The advantages and features provided by one or more embodiments of the invention will become more fully understood from the detailed description given hereinbelow and the appended drawings which are given by way of illustration only, and thus are not intended as a definition of the limits of the present invention, wherein:

FIG. 1 is a schematic configuration of a dynamic image display system;

FIG. 2 is a block diagram showing functional configuration of an image display apparatus;

FIG. 3 is a flowchart of a comparison display process performed by a controller shown in FIG. 2;

FIG. 4 schematically illustrates how a reference frame image is enlarged through the comparison display process;

FIG. 5A shows a to-be-interpreted dynamic image and a reference dynamic image that are enlarged at the same enlargement ratio and displayed side by side for comparative image interpretation; and

FIG. 5B shows the to-be-interpreted dynamic image and the reference dynamic image that are displayed side by side for comparative image interpretation, wherein the reference dynamic image has been enlarged through the comparison display process in this embodiment.

### DETAILED DESCRIPTION OF THE EMBODIMENTS

Hereinafter, an embodiment of the present invention is described with reference to the drawings. However, the scope of the present invention is not limited to the disclosed embodiment.

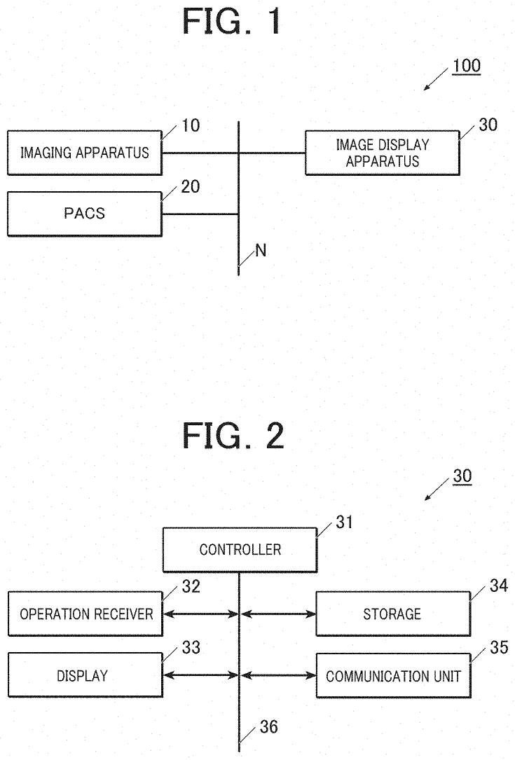

FIG. 1 shows a schematic configuration of a dynamic image display system 100 in an embodiment of the present invention.

As shown in FIG. 1, the dynamic image display system 100 includes an imaging apparatus 10, a picture archiving and communication system (PACS) 20, and an image display apparatus 30. These apparatuses are connected to send/receive data to/from each other via a communication network N, such as a local area network (LAN) or a wide area network (WAN). The apparatuses constituting the dynamic analysis system 100 conform to the digital image and communications in medicine (DICOM) standard and communicate with one another in accordance with the DICOM.

The imaging apparatus 10 performs dynamic imaging. Dynamic imaging refers to continuously taking images of a subject in motion and thereby obtaining a dynamic image that consists of multiple frame images showing the dynamic state of the subject. For example, the imaging apparatus 10 obtains a dynamic image of a subject by repetitively emitting pulsed radiation (e.g., X-rays) to the subject at predetermined time intervals (pulse emission) or continuously emitting radiation without a break to the subject at a low dose rate (continuous emission). The imaging apparatus 10 associates each of the obtained frame images with header information and sends the frame images to the PACS 20. The header information includes, for example, (i) patient information, such as the patient ID, patient name, height, weight, age, and sex, (ii) examination information, such as the examination ID, examination date, and imaged region, and (iii) a frame number showing the imaging order of the frame image.

In this embodiment, dynamic images are obtained in the DICOM format but may be obtained in other moving-image formats, such as MGEG or MP4.

The PACS 20 is a server that stores and manages medical images including dynamic images generated by the imaging apparatus 10. The PACS 20 includes a database to store the medical images in association with patient information, examination information, frame numbers, case names, and other information. For example, the PACS 20 sends, to the image display apparatus 30, a list of examinations that have been done by the imaging apparatus 10. When an examination is specified at the image display apparatus 30, the PACS 20 retrieves medical images of the specified examination and sends the images to the image display apparatus 30. Further, when a search condition is specified at the image display apparatus 30, the PACS 20 retrieves medical images that meet the search condition from the database and sends the images to the image display apparatus 30.

The image display apparatus 30 is a computer that reads, in response to the operation performed by a user (e.g., doctor), medical images stored in the PACS 20 and displays the medical images for diagnosis.

FIG. 2 shows a functional configuration of the image display apparatus 30.

As shown in FIG. 2, the image display apparatus 30 includes a controller 31 (hardware processor), an operation receiver 32, a display 33, a storage 34, and a communication unit 35. These components are connected via a bus 36.

The controller 31 includes a central processing unit (CPU), a read only memory (ROM), and a random access memory (RAM). The controller 31 centrally controls processing operation of the components of the image display apparatus 30. More specifically, the CPU of the controller 31 reads various processing programs stored in the ROM, loads them into the RAM, and performs various processes in cooperation with the programs.

The operation receiver 32 includes: a keyboard with character input keys, number input keys and various function keys; and a pointing device, such as a mouse. In response to a key being pressed on the keyboard or the mouse being operated by the user, the operation receiver 32 outputs, to the controller 31, a press signal of the key or an operation signal of the mouse as an input signal.

The display 33 includes a monitor, such as a cathode ray tube (CRT) or a liquid crystal display (LCD). The display 23 displays various screens in accordance with the instruction of display signals input by the controller 31.

The storage 34 includes a hard disk drive (HDD) and/or a nonvolatile semiconductor memory and stores various kinds of data.

The communication unit 35 includes a network interface. The communication unit 35 sends and receives data to and from external apparatuses connected over the communication network N.

Next, operation of the image display apparatus 30 is described.

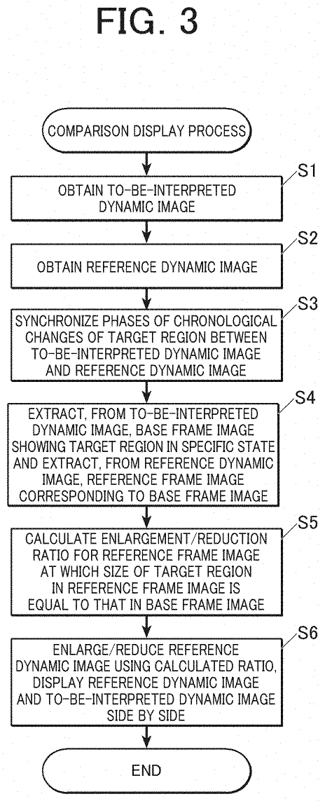

FIG. 3 is a flowchart showing a comparison display process performed by the controller 30. The process is performed when an examination (an image to be interpreted) is selected with the operation receiver 32 from the list of examinations displayed on the display 33, for example. The process is performed through software processing by the CPU of the controller 30 in cooperation with the programs stored in the ROM.

The controller 31 requests, via the communication unit 35, the PACS 20 to send a to-be-dynamic image that corresponds to the examination selected with the operation receiver 32 and obtains the to-be-interpreted dynamic image as a base dynamic image (Step S1).

The controller 31 further obtains a reference dynamic image that is to be compared with the to-be-interpreted dynamic image (Step S2).

For example, the user (e.g., doctor) operates the operation receiver 32 to specify the search condition of the reference dynamic image to be compared with the to-be-interpreted dynamic image. The controller 31 sends the specified search condition to the PACS 20 via the communication unit 35 and obtains, from the PACS 20, the dynamic image(s) that meets the specified condition as the reference dynamic image.

The reference dynamic image is, for example, a dynamic image of a case similar to the case of the to-be-interpreted dynamic image. The reference dynamic image may be a dynamic image that shows the same imaging region as the to-be-interpreted dynamic image and that shows a normal or abnormal case.

Further, two or more reference dynamic images may be obtained.

The controller 31 synchronizes the phases of chronological changes of the target region between the to-be-interpreted dynamic image and the reference dynamic image (Step S3).

The target region is a target of the function diagnosis. The target region may be determined beforehand in association with the imaging region or may be specified by the user using the operation receiver 32.

In Step S3, the controller 31 extracts, from each of the frame images constituting the to-be-interpreted dynamic image and the reference dynamic image, an area corresponding to the target region to obtain the size of the target region. For each of the to-be-interpreted dynamic image and the reference dynamic image, the controller 31 generates a waveform of chronological changes in size of the target region. The controller 31 determines whether or not the phase of the waveform of the to-be-interpreted dynamic image synchronizes with the phase of the waveform of the reference dynamic image. When determining that these phases do not synchronize, the controller 31 shifts at least one of the waveforms such that the phases synchronize. The controller 31 then shifts, along the time axis, frame images of the dynamic image of which waveform has been shifted.

For example, assume that the to-be-interpreted dynamic image and the reference dynamic image are dynamic chest images for making a diagnosis on the function of the lungs. For each of the to-be-interpreted dynamic image and the reference dynamic image, the controller 31 extracts the lungfield region from each of the frame images constituting the dynamic image to obtain a feature quantity. The feature quantity indicates the size of the lungfield region. For each of the to-be-interpreted dynamic image and the reference dynamic image, the controller 31 generates a waveform that shows chronological changes of the obtained feature quantity. The controller 31 determines whether or not the phase of the waveform of the to-be-interpreted dynamic image synchronizes with the phase of the waveform of the reference dynamic image. When determining that these phases do not synchronize, the controller 31 shifts at least one of the waveforms so that the phases synchronize. The controller 31 then shifts, along the time axis, frame images of the dynamic image of which waveform has been shifted.

Herein, expansion of the lungfield region from each frame image may be done according to any method. For example, the controller 31 performs discriminant analysis on the basis of a histogram showing signal values (densities) of pixels to obtain a threshold. The controller 30 then extracts a region having signals higher than the threshold as a lungfield region candidate. The controller 31 performs edge detection around the border of the extracted lungfield region candidate. The controller 31 extracts, from small blocks around the border, points at which the edge is the maximum. The controller 31 thus extracts the border of a lung field region.

The feature quantity that indicates the size of the lungfield may be, for example, the area of the lungfield region. The area of the lungfield region can be determined by counting pixels within the extracted lungfield region, for example. The feature quantity that indicates the size of the lungfield may also be the distance between the apex of the extracted lungfield region and the diaphragm (the position/coordinate of the diaphragm in the width direction of the image).

When the cycles of chronological changes of the target region are different between the to-be-interpreted dynamic image and the reference dynamic image, the controller 31 may synchronize the phases between these dynamic images such that the dynamic images each show a specific frame image corresponding to the target region in a specific state at the same timing. For example, the controller 31 may shift frame images of at least one of the dynamic images along the time axis such that the dynamic images each show a frame image corresponding to the maximum/minimum point of the waveform at the same timing.

Alternatively, the controller 31 may first synchronize the cycles of chronological changes of the target region between the to-be-interpreted dynamic image and the reference dynamic image and then synchronize the phases of these dynamic images. Assume that the chronological change cycle of the target region in the reference dynamic image is shorter than that in the to-be-interpreted dynamic image, for example. In the case, the controller 31 adds frame images to the reference dynamic image at regular intervals in the time direction (upsamples the reference dynamic image) such that the cycle of the target region in the reference dynamic image is the same as that in the to-be-interpreted dynamic image. Pixel values (densities) of added frame images can be determined by interpolation processing on the basis of corresponding pixel values of frame images of the original dynamic image, for example. Assume that the chronological change cycle of the target region in the reference dynamic image is longer than that in the to-be-interpreted dynamic image. In the case, the controller 31 reduces the number of frame images (deleates frame images) of the reference dynamic image at regular intervals in the time direction (downsamples the reference dynamic image) such that the cycle of the target region in the reference dynamic image is the same as that in the to-be-interpreted dynamic image.

The controller 31 extracts, among the frame images constituting the to-be-interpreted dynamic image, a base frame image that corresponds to the timing at which the target region is in a specific state. The controller 31 also extracts, among the frame images constituting the reference dynamic image, a reference frame image that corresponds to the base frame image (Step S4).

For example, the controller 31 extracts, from the to-be-interpreted dynamic image, a frame image that corresponds to the timing at which the size of the target region is largest/smallest as the base frame image, and extracts, from the reference dynamic image, a frame image that corresponds to the base frame image as the reference frame image. The controller 31 thus extracts, from the reference dynamic image, the reference frame image that shows the same timing as the base frame image in the cycle of chronological changes of the size of the target region.

For example, among frame images constituting the to-be-interpreted dynamic image, the controller 31 extracts a frame image that corresponds to the maximal inspiratory level as the base frame image. The controller 31 then extracts, among the frame images constituting the reference dynamic image, a frame image that corresponds to the base frame image, or more specifically, a frame image that corresponds to the maximal inspiratory level. The frame images at the maximal inspiratory level show the lungfield region in its maximum size.

The base frame image may not be the image at the maximal inspiratory level but may be, for example, the image at the maximal expiratory level.

The controller 31 calculates the enlargement/reduction ratio that equalizes the size of the target region in the reference frame image with the size of the target region in the base frame image (Step S5).

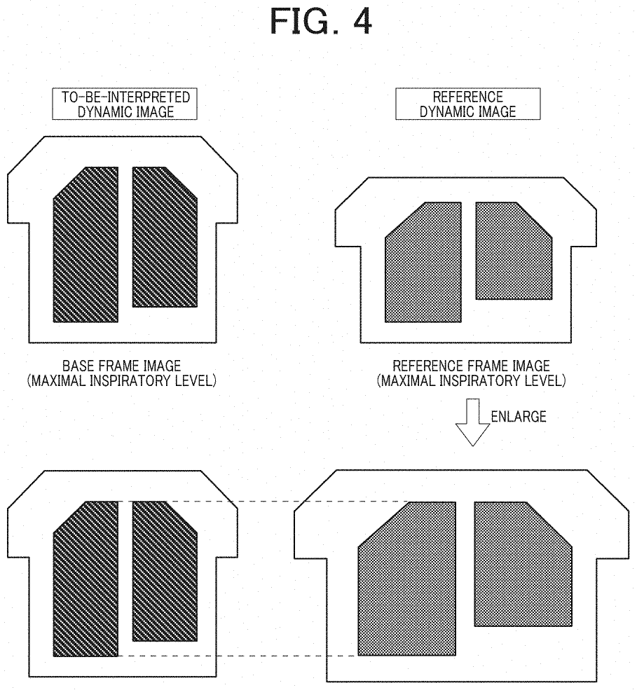

FIG. 4 shows the base frame image of the to-be-interpreted dynamic image and the reference frame image of the reference dynamic image at the maximal inspiratory level. In FIG. 4, the lungfield region (right lung region) at the maximal inspiratory level in the reference frame image is smaller than that in the base frame image. The controller 31 calculates the enlargement ratio that equalizes the size of the lungfield region (right lung region) in the reference frame image with the size of the lungfield region in the base frame image.

In the present description, “equalize” includes the meaning of “substantially equalize” as well as the meaning of “exactly equalize”.

The controller 31 enlarges/reduces each of the frame images constituting the reference dynamic image by using the enlargement/reduction ratio calculated in Step S5, and displays, on the display 33, the enlarged/reduced reference dynamic image and the to-be-interpreted dynamic image side by side such that the frame images of the respective dynamic images are sequentially switched (Step S6). The controller 31 then ends the comparison display process.

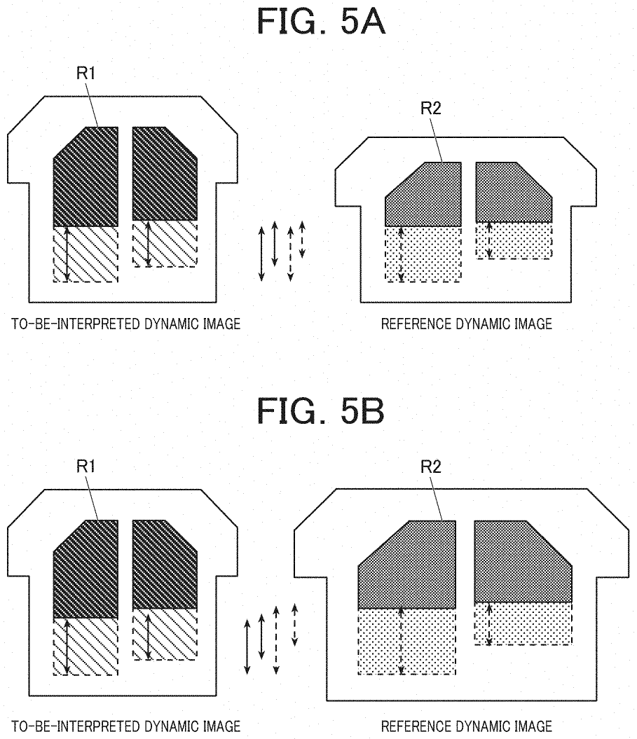

FIG. 5A and, FIG. 5B show the to-be-interpreted dynamic image and the reference dynamic image displayed side by side. In FIG. 5A and FIG. 5B, the size of the target region in the reference dynamic image is smaller than that in the to-be-interpreted dynamic image.

FIG. 5A shows the to-be-interpreted dynamic image and the reference dynamic image that are enlarged at the same enlargement ratio and displayed side by side. In FIG. 5A, the amount of expansion/contraction of the right lung R1 in the to-be-interpreted dynamic image (shown by solid arrow) appears to be the same as the amount of expansion/contraction of the right lung R2 in the reference dynamic image (shown by dotted arrow).

FIG. 5B shows the to-be-interpreted dynamic image and the reference dynamic image that are displayed side by side. In FIG. 5B, the reference dynamic image has been enlarged according to the above comparison display process. More specifically, each of the frame images constituting the reference dynamic image has been enlarged at the enlargement ratio that equalizes the size of the lungfield region at the maximal inspiratory level in the reference frame image with the size of the lungfield region at the maximal inspiratory level in the base frame image in the to-be-interpreted dynamic image. As shown in FIG. 5B, after the comparison display process, the expansion/contraction amount of the right lung R1 in the to-be-interpreted dynamic image (shown by solid arrow) is smaller than the expansion/contraction amount of the right lung R2 in the reference dynamic image (shown by dotted arrow). More specifically, the change rate of the lungfield area of the right lung R1 is smaller than that of the right lung R2. FIG. 5B thus shows that the patient of the to-be-interpreted dynamic image has a lower respiratory function than the patient of the reference dynamic image.

As described above, according to the comparison display process, the reference dynamic image is enlarged/reduced such that the reference dynamic image and the to-be interpreted dynamic image show the target region at a timing of a specific state in the same size, and the enlarged/reduced reference dynamic image and the to-be-interpreted dynamic image are displayed side by side. This allows an image interpreter (e.g., doctor) to easily compare change rates of the size of the target region even when the size of the target region is different between the to-be-interpreted dynamic image and the reference dynamic image. Accordingly, the image interpreter can make an appropriate diagnosis on the function of the target region.

When the case name of the to-be-interpreted dynamic image is identified after the image interpretation and is input with the operation receiver 32, the controller 31 sends the case name and the examination ID of the to-be-interpreted dynamic image to the PACS 20 via the communication unit 35. The PACS 20 stores the case name in association with the dynamic image that corresponds to the sent examination ID.

As described above, according to the image display apparatus 30, the controller 31 enlarges/reduces at least one of multiple dynamic images at an enlargement/reduction ratio that equalizes the size of the target region in a specific state among the multiple dynamic images, and causes the display 33 to display the multiple dynamic images

Accordingly, an image interpreter can smoothly and appropriately make a diagnosis on the function of the target region on the basis of multiple dynamic images displayed side by side for comparison.

The above embodiment is a preferred example of the image display apparatus according to the present invention and does not limit the present invention.

For example, in the above comparison display process, all the dynamic images are displayed and compared, and at least one of the dynamic images is enlarged/reduced such that the sizes of the target region in a specific state are equal among all the dynamic images. However, all the displayed dynamic images may not have to be compared. In such a case, among the displayed dynamic images, at least two dynamic images may be compared. At least one among the at least two dynamic images may be enlarged/reduced such that the sizes of the target region in the specific state are equal among the at least two dynamic images. More specifically, the controller 31 enlarges/reduces at least one among the at least two dynamic images at the enlargement/reduction ratio that equalizes the size of the target region in the specific state among the at least two dynamic images, and causes the display 33 to display the at least two dynamic images.

Further, in the above embodiment, the to-be-interpreted dynamic image is set as the base dynamic image, and the reference dynamic image to be compared with the base dynamic image is enlarged/reduced at the enlargement/reduction ratio that equalizes the size of the target region in the reference dynamic image with the size of the target region in the to-be-interpreted dynamic image. Alternatively, the reference dynamic image, which is compared with the to-be-interpreted dynamic image, may be set as the base dynamic image. In the case, the to-be-interpreted dynamic image may be enlarged/reduced at the enlargement/reduction ratio that equalizes the size of the target region in the to-be-interpreted dynamic image with the size of the target region in the reference dynamic image.

Further, the comparison display process may be performed by the controller of the PACS 20 in cooperation with the CPU and the program, and the to-be-interpreted dynamic image and the reference dynamic image may be displayed on the display 33 of the image display apparatus 30.

Further, although a semiconductor memory and/or HDD are used in the above embodiment as a computer-readable medium that stores the programs for performing various kinds of processing, the computer readable medium is not limited thereto. As the computer readable medium, a portable storage medium, such as a CD-ROM, can also be used. Further, as a medium to provide data of the programs via a communication line, a carrier wave can be used.

The detailed configurations/components and operation of the components constituting the image display apparatus can also be appropriately modified without departing from the scope of the present invention.

Although embodiments of the present invention have been described and illustrated in detail, the disclosed embodiments are made for purposes of illustration and example only and not limitation. The scope of the present invention should be interpreted by terms of the appended claims.

What is claimed is:

1. An image display apparatus comprising:
a display that displays multiple dynamic images; and
a hardware processor, wherein
the multiple dynamic images include at least two dynamic images and show a target region, and
the hardware processor enlarges/reduces at least one of the at least two dynamic images at an enlargement/reduction ratio that equalizes a size of the target region in a specific state among the at least two dynamic images, and causes the display to display the at least two dynamic images.

2. The image display apparatus according to claim 1, wherein
the at least two dynamic images include a base dynamic image and a reference dynamic image, and
the hardware processor enlarges/reduces the reference dynamic image at the enlargement/reduction ratio that equalizes the size of the target region in the specific state in the reference dynamic image with the size of the target region in the specific state in the base dynamic image.

3. The image display apparatus according to claim 2, wherein
the base dynamic image is a dynamic image to be interpreted, and
the reference dynamic image is a dynamic image of a case similar to a case of the base dynamic image.

4. The image display apparatus according to claim 2, wherein the hardware processor
synchronizes phases of chronological changes of the target region between the base dynamic image and the reference dynamic image,
extracts, among frame images constituting the base dynamic image, a base frame image that shows the target region in the specific state,
extracts, among frame images constituting the reference dynamic image, a reference frame image that corresponds to the base frame image, and
enlarges/reduces each of the frame images constituting the reference dynamic image at the enlargement/reduction ratio that equalizes the size of the target region in the reference frame image with the size of the target region in the base frame image.

5. The image display apparatus according to claim 2, wherein
the multiple dynamic images are dynamic chest images,
the target region in the specific state is a lungfield at a maximal inspiratory level, and
the hardware processor enlarges/reduces each of frame images constituting the reference dynamic image at the enlargement/reduction ratio that equalizes the size of the lungfield at the maximal inspiratory level in a frame image of the reference dynamic image with the size of the lungfield at the maximal inspiratory level in a frame image of the base dynamic image.

6. A non-transitory computer-readable storage medium storing a program that causes a computer to function as a hardware processor that:
among multiple dynamic images that are to be displayed on a display and that include at least two dynamic images and that show a target region, enlarges/reduces at least one of the at least two dynamic images at an enlargement/reduction ratio that equalizes a size of the target region in a specific state among the at least two dynamic images, and
causes the display to display the at least two dynamic images.
